(19) United States
(12) Patent Application Publication
(10) Pub. No.: US 2007/0104840 A1
(43) Pub. Date: May 10, 2007

(54) METHOD AND SYSTEM FOR THE DETERMINATION OF PALATABILITY

(76) Inventors: Michael G. Singer, Harrisville, MI (US); John D. Kutzko, Nokomis, FL (US)

Correspondence Address: Irving M. Weiner;Weiner & Burt, P.C., P.O. Box 186, 635 N. US-23, Harrisville, MI, 48740, US

(21) Appl. No.: 11/548,003

(22) Filed: Oct. 10, 2006

Related U.S. Application Data
(63) Continuation-in-part of application No. 11/386,016, filed on Mar. 18, 2006.
(63) Continuation-in-part of application No. 10/701,004, filed on Nov. 4, 2003, now Pat. No. 7,003,346.
(63) Continuation-in-part of application No. 09/848,242, filed on May 3, 2001, now Pat. No. 6,587,715.
(60) Provisional application No. 60/826,774, filed on Sep. 25, 2006.
(60) Provisional application No. 60/827,698, filed on Sep. 30, 2006.
(60) Provisional application No. 60/594,200, filed on Mar. 18, 2005.
(60) Provisional application No. 60/424,828, filed on Nov. 8, 2002.

(51) Int. Cl. G01N 33/02

(52) U.S. Cl. 426/231

(57) ABSTRACT

A method and system to determine palatability (tenderness, juiciness, and flavor) of Eve foodstuffs (meat, fish, fowl, ft and vegetables) including the steps of, utilizing bioelectrical impedance analysis in a biological subject model for measurement and composition analysis; and a system of using the results of the utilizing step procedure to illustrate an objective scale of palatability; a ‘Palatability Index’.

### CROSS-REFERENCE TO RELATED APPLICATIONS

The present application claims priority from and is continuation-in-part of: 1) U.S. Provisional Patent Application 60/826,774 filed Sep. 25, 2006; 2) U.S. Provisional Patent Application 60/827,698 filed Sep. 30, 2006; and 3) U.S. patent application Ser. No. 11/386,016 filed Mar. 18, 2006, which in turn is a continuation-in-part of and claims priority from U.S. Provisional Patent Application 60/594,200 filed Mar. 18, 2005, which in turn is a continuation-in-part of and claims priority from U.S. patent application Ser. No. 10/701,004 filed Nov. 4, 2003, now U.S. Pat. No. 7,003,346, which in turn is based on and claims priority from U.S. Provisional Patent Application Ser. No. 60/424,828 filed Nov. 8, 2002, which is a continuation-in-part of U.S. patent application Ser. No. 09/848,242 filed May 3, 2001, now U.S. Pat. No. 6,587,715. The complete disclosure of the aforementioned patent applications and patents are incorporated herein by reference thereto.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

The present invention relates generally to a method and system of determining palatability of a foodstuff biological entity including at least a portion of a live or previously-live organism,

More particularly, an embodiment of the invention relates to the aforementioned method and system is comprised to determine the optimal aging of meat.

After safety (which is often assumed), consumers believe palatability to be the single most important component of meat and foodstuff quality. This is confirmed by the relationship between the price of a cut of meat or other foodstuffs and its palatability. While a subjective concept there are objective scaling aspects of the components of palatability; juiciness (water content), tenderness (density of cell mass and membranes) and flavor (changes in juiciness and tenderness that while subjective can be measured and scaled and offered to an individual to select a score for them (a personal preference) for consistency in product selection) Inconsistency in palatability has been identified as one of the major problems facing the foodstuffs industries most notably in those more likely perishable and predominantly in the meats and particularly the beef industry. Uniformity, excessive fatness, and inadequate tenderness/palatability are all primary quality concerns of the beef industry.

Market surveys have reported that consumers were dissatisfied with the eating quality (palatability) of beef prepared at home nearly 25% of the time. Consumers that returned foodstuffs they were not satisfied with reported the source of their dissatisfaction was due to palatability troubles nearly 80% of the time. The actual extent of the palatability problem is more expansive and startling, as less than one-percent of discontented customers actually complains or returns the non-palatable product. This happens despite the technology that has been developed to improve the consistency and quality of palatability.

For instance the beef industry relies on the USDA quality grading system to segment carcasses into groups based on varying levels of expected meat palatability. Results of numerous investigations of the relationship between marbling and beef palatability indicate that, although there is a positive relationship between marbling degree and tenderness, juiciness, and flavor, this relationship is limited at best. There are too many carcasses with tender meat that are discounted unnecessarily and sold for less than their potential value and too many with tough meat that are not discounted under the current USDA quality grading system used for a basis of palatability classification is neither accurate nor uniform. Data collected in various animal research centers clearly point out that estimates of raw and cooked steak chemical composition are unrelated to palatability.

Inconsistency in the palatability of foodstuffs is multifactorial and may result from several factors external and internal, individually and collectively to include its genetic make-up, constituent composition (micro (minerals) and macro (fats, proteins, carbohydrates))(of note: it is not the intent of the present invention to assess micro and/or macro nutrient composition only their potential (and that of any other factor) impact on the volume and distribution of fluids, tissues and cells, as well as the electrical health and vitality of the cells and membranes and relate them to palatability), nutrients received (type and/or timing) during growing, method of harvest, time from harvest to consumption, transportation methods/conditions, storage, aging, methods of display, dispensing and preparation. However the individual and combined, internal and/or external factors and effects will impact and result in evidence impacting the cellular level make-up of the volume and distribution of fluids, tissues and cells, as well as the electrical health and vitality of the cells and membranes.

In meats for instance, particularly beef several characteristics are considered to assess palatability to include, genetic make-up and breeding, feed, fat content, and time from harvest to consumption (aging). A variety of methods are employed to enhance the palatability of beef; the injection of chemicals and minerals (calcium activated tenderization), mechanical and electrical ‘treatment’. Indirect methods of predicting palatability include carcass traits (‘marbling’), analysis of the enzymes associated with proteolysis (calpastatin activity) and genetic approaches (candidate genes, gene mapping); used both individually and in combination. Whatever the means to impact the palatability of the beef a standardized ‘sheer force’ test is used to measure, test and document the tenderness component

In meats, the changes that take place post-mortem in the conversion of muscle to meat are based upon natural enzymes (proteases) that breakdown proteins (proteolysis) in the muscle fibers (myofibrils) is called fragmentation. It is the process of fragmentation that improves tenderness. This ‘aging’ process begins immediately post-mortem and progresses at various rates (based upon species and cut) from three to twenty-one (or more as some preferences and reports document ninety days of aging) days reaching optimal values sometime during that continuum. It should also be noted that the ‘Aging’ process may progress beyond palatability and becomes detrimental with microbial over-growth and distinct negative flavor and even adverse health events possible. Aging may be carried out ‘dry’; without covering or packaging in a controlled environment (temperature, humidity and airflow) or ‘wet’; vacuum packaged in plastic. While both methods are effective, each has significant variations in flavor and cost of the end product. The timing of the changes to reach optimal palatability is subjective with no direct analysis method available until the present invention which illustrates the changes of the volume and distribution of fluids, tissues and cells, as well as the electrical health and vitality of the cells and membranes throughout the proteolysis process. Correlation of the measured electrical values enables an objective and subjective scale to be established and used for the optimization of palatability and the timing of aging, palatability based pricing, and controls.

The prior, but not necessarily relevant, art is exemplified in the following U.S. patents and patent applications.

U.S. Pat. No. 4,985,125, issued in 1991, to Watanabe, et al; entitled; “Method for Detecting Meat Freshness Using a Biosensor”, in which is described, “A biosensor comprising a main body and a built-in sensing electrode provided therein, wherein said sensing electrode is provided with a sensing part to which a bur solution can be constantly fed and said main body is provided with an inlet which is brought into direct contact with a specimen to thereby incorporate the molecule to be assayed from said specimen there through, said inlet being covered with a membrane through which the molecule to be assayed can permeate. This biosensor enables a specified molecule contained in a specimen to be rapidly and conveniently assayed without requiring any pretreatment of the specimen.”

U.S. Pat. No. 5,088,822, issued in 1992 to Kanda, entitled; “Meat Freshness Measuring Apparatus”, in which is described; “A meat freshness measuring apparatus measures the freshness of meat by detecting changes in a pigment contained in the meat. For this purpose, the meat is exposed to rays of light of different wavelengths applied from a light source, and the rays obtained from the meat are separated into spectra for the respective wavelengths, which are received by a photoelectric conversion element. Spectrum data for each wavelength are amplified and then the amplified data are converted to a digital signal by an A/D converter, whereby the digital signal is stored in a RAM. Based on the stored spectrum data and using a prescribed equation of calculation, a content of the pigment in the meat is calculated and outputted by a CPU.

U.S. Pat. No. 5,788,643, issued in 1998 to Feldman entitled “Process for Monitoring Patients with Congestive Heart Failure”, in which is described “In a process for monitoring patients with chronic congestive heart failure, a high frequency current is passed between electrodes applied to two limbs of a patient. The current, voltage and phase angle between the measured current and voltage are measured to enable the calculation of congestive heart failure (CHF) indicia values. The calculated CHF indicia values are then compared with baseline values established when the patient is in a known, stable condition. Intervention is initiated if the differences between the calculated CHF indicia values and the baseline values are outside of established tolerances. The CHF indicia values may include resistance, reactance, impedance, total body water and extracellular water. Moreover, the CHF indicia values may include a figure of merit indicative of the hydration status of the patient.

U.S. Published patent application 2004/0014235, published in 2004 by Kelly, et al, entitled “Polymeric Food Spoilage Sensor”, in which is described, “A polymeric food spoilage sensor comprises a polymer containing a polyazamacrocyclic transition metal complex. The complex selectively binds biogenic amines, such as cadaverine, putrescine and histamine, which are released by food spoilage microorganisms. The polymer undergoes a detectable color change upon exposure to biogenic amine, thus indicating that food spoilage has probably occurred. In one embodiment, the polymer is molecularly imprinted with the biogenic amine to impart selective binding affinity. The polymer is easily incorporated in common food containers and can be employed in fiber optic detection devices.”

U.S. Published patent application 2005/0058751, published in 2005 by Brotsky et al., entitled; “Yield and Shelf Life for Meats”, in which is described an; “The invention is directed to a process for treating meat to achieve higher yield, without negatively affecting the appearance of the meat. The process comprises the steps of 1) treating the meat, preferably by injection, with a higher pH phosphate solution having a pH of above pH 6.0 and 2) after treating the meat with the higher pH phosphate solution, contacting the meat, preferably by dipping or spraying, with a lower pH solution of preferably less than about pH 5.6.”

The desire of the present invention is to avoid criticism of conventional methods and techniques, and to provide a novel method for determination of the palatability of live foodstuffs.

### SUMMARY OF THE INVENTION

The term ‘live’ foodstuffs as used herein means any and all living organisms to include; meats, fish, fowl, fruits and vegetables.

The term ‘biological entity’ as used herein means any and all portions, parts or whole of a live or previously-live organism.

The term ‘subject’ as used herein means that portion, segment, ‘cut’ or whole biological entity studied.

The term ‘electrode scheme’ as used herein means any and all configurations utilized to introduce and measure the electrical signal and corresponding voltage drop by placement on the subject's surface, around said surface, into said subject and/or through placing said subject onto the electrode configuration singularly or as part of another appliance.

The term ‘average’ as used herein means the product of the statistically valid sample size number divided into the measured values.

The term ‘normal’ as used herein means the product of the average peculiar to and comprised of but not limited to a defined group; age, gender, species, or cut.

The term ‘optimal’ as used herein means the best or most favorable value; which may be obtained subjectively individually or collectively or it may be obtained objectively as compared to a ‘criterion’ or ‘gold-standard’ designated and agreed upon by professional, experts and those ‘experienced’ in the field of endeavor and by personal selection of a value on that objective scale an individual may express and select their personal optimal value.

The term ‘individual’ as used herein means those findings peculiar to a single subject or to a uniformly collective group of individual subject's assigned to a group based upon a preponderance of similar and agreed upon characteristics such as but not limited to; genus, species, cut, breed, or other such recognized characters of physicality and composition.

The term ‘meat’ as used herein means bovine (Bos), porcine, lamb (Ovis Aries), buffalo, bison camel, goat (Capra Hircus) equine, donkey, Hama, reindeer and yak.

The terms ‘fowl’ or ‘poultry’ as used herein means; chicken, turkey, duck, geese, guinea fowl and swan.

The term ‘external appliance’ as used herein is comprised of but not limited to scales, refrigerators, display, and/or packaging materials, methods, device or systems and portable temperature controlled appliances, and cooking appliances.

The term ‘Palatability Index’ as used herein are the objective results scaled to the characteristics of the foodstuff and reported in priority of importance; safe versus unsafe and then as varying degrees of palatability and used to support subjective decisions of producers, purveyors, merchants preparers, and consumers of said foodstuffs for the purposes of preference, pricing, acquisition and safety and health.

The present invention provides a method and system of determining palatability of a foodstuff biological entity including at least a portion of a live or previously-live organism, comprising the steps of: subjecting said foodstuff biological entity to bioelectrical impedance analysis for measurement and composition analysis; and utilizing results of said subjecting step to illustrate an objective scale of palatability of said biological entity.

The present invention also provides a method for determining the palatability of a biological entity, grossly in terms of safe or not safe and then more precisely as related to juiciness, tenderness and flavor. Changes of palatability of said biological entity, and/or timing of optimal palatability, loss of the palatability of said biological entity and/or illustrating an objective scale of palatability from which a producer, purveyor, merchant and/or consumer may objectively and subjectively apply their institutional, commercial or individual tastes and select from said scale their preference, comprising the steps of: providing normal, average, optimal and individual measured values of resistance, reactance, capacitance and phase angle, of the sample subject studied of the biological entity; measuring initial values of resistance, reactance, capacitance and phase angle, of the sample subject biological entity; taking measurements of resistance, reactance, capacitance and phase angle, at predetermined intervals of time based upon the characteristics and proposed utilization of the individual subject; recording said measurements; comparing initial values of said measurements to normal values of said measurements and to serially measure values of said measurements; and determining, from said comparison steps, hallmarks of palatability of said biological entity, said progression and rate of changes in palatability to a zenith, nadir or loss of the palatability of said biological entity, to a specific individual ‘Palatability Index’ value which may be reported and found as the inherent average, normal, optimal and/or individual characteristics of said biological entity or portion thereof.

### DETAILED DESCRIPTION OF THE INVENTION

The invention provides a method and system to obtain and use the measured values and products of bioelectrical impedance analysis (BIA) as an objective means to equivalently illustrate electrically, various physiological characteristics, and upon which characterization the palatability of foodstuffs can be objectively and subjectively described and compared and practically utilized.

The method of BIA measurement may comprise various configurations so as to accommodate the diversity of foodstuffs so measured to the extent that the interface with the foodstuff (electrode array/scheme, electrical power management (frequencies, current and voltages)) and circuit models (series and/or parallel) may be varied as such to incorporate the subject foodstuff within the controlled electrical circuit or field of the BIA measurement comprised in such manner as to complete said measurement.

The interfaces for electrode array/scheme may be comprised of; placement of the studied foodstuff within a generated electrical field array, on an electrode scheme array, placing the electrode array about around or as comprised in such configuration as to measure ‘capture’, characterize and illustrate the unique geometry and traits of the subject foodstuff in its entirety or as possible the electrode scheme and array may be introduced directly into the study subject foodstuff, and/or that such electrical power management configurations may be comprised of fixed or variable frequencies, currents and voltages and circuit models (series and/or parallel) and that the measured and calculated values be comprised of such values and sampling rates to adequately capture, characterize and illustrate the unique geometry and traits of the subject foodstuff in its entirety.

The electrical signals utilized to measure and calculate the impedance, resistance, reactance, capacitance and phase angle may be comprised of multiple schemes based upon the type and geometry of the foodstuff; a mono or singular frequency, multiple frequencies, or a spectroscopic illustration across a segment or band of frequencies.

The measured and calculated electrical values comprised of impedance, resistance, reactance, capacitance and phase angle are related to the comprised physiological values of fluid; volume and distribution, the cell mass; volume, character and membrane vitality as related to the unique and inherent characteristics palatability (flavor, juiciness and tenderness) of the studied subject foodstuff and reported in such a manner as to provide a basis for objective assessments and subjective interpretation of said comprised values for foodstuff product; safety grading, pricing, handling, management and disposition.

Thus, the present invention provides a method and system for the use of bioelectrical impedance analysis (BIA) in the electrical measurement of a biological equivalent model of ‘live’ foodstuffs or ‘biological entities’ to provide an objective assessment and scale of palatability to include safety, freshness, juiciness, flavor and tenderness as related to the characteristics, volume and distribution of fluids, tissues and cells as well as the electrical vitality of cells and cell membranes through the measurement of impedance (Z), resistance (R), reactance (Xc), capacitance (Cp) and the calculation of phase angle (Pa) at a fixed or variable electrical frequency, current and voltage through a tetrapolar electrode scheme placed on, around and/or in or with the subject placed upon the array or by placing the study subject within a electrical field or a portion thereof by placing said foodstuff biological entity or a portion thereof onto an electrode configuration singularly or as comprised as part of an external appliance; such as part of a scale; refrigerator or a portable temperature controlling device, packaging or display, the study subject as measured individually; compared to normal, average and optimal values and as tracked serially over time and compared to changes from the initial measurement.

More specifically the present invention provides a method and system for determining the palatability of a portion or whole live or previously live foodstuff such as a meat, fish, fowl, fruit or vegetable, to grade its characteristics (palatability), quality and salability, and to support decisions regarding its disposition, preparation and presentation and cost and consumption.

The methods of the present invention can utilize a modification of the body composition analyzer disclosed in U.S. Pat. No. 5,372,141, the entire contents of which are incorporated herein by reference thereto. Such modification may include, but not be limited to, impedance measuring instrumentation capable of measuring impedance, resistance and reactance for the calculation of capacitance and phase angle from selected singular or mono-frequency, multiple frequencies and/or impedance spectroscopic analysis or changes in current, power and voltage.

In accordance with the present invention, utilization of BIA in a biological model provides an objective assessment of the study subject's (whole or section of the biological entity) volume and distribution of fluids, tissues and cells, as well as the electrical health and vitality of the cells and membranes.

The characteristics of BIA include precision, accuracy, feasibility and economy. BIA may be applied to any subject whole or an area of representative sample or interest to be studied and examined for palatability; a section thereof, regionally, or to the whole biological entity. It is non-offensive, causing no harm. It may be repeated freely, as desired to capture various dynamic changes unique to the variety of live foodstuffs (biological entities), to illustrate initial values and change over time so that progression of conditions can be monitored and changes that effect palatability determined. The specific value of BIA is in its precision of measurement and the significance of the electrically measured products illustration of the biological foodstuff entities equivalent physiological variables of fluid, tissue and cells volume and distribution, cell membrane volume and vitality, derivative values initially and comparison to average, optimal, normal, and subsequent individual values and changes serially over time.

Based upon the individual genus, type; species, ‘cut’ or sample of the biological foodstuff entity, palatability is determined by the baseline values, and changes thereto (rate, zenith and nadir) of the measured and calculated values initially and over time. The properties of the electrical values directly relate to biological equivalents such as; measured R is inverse to water content (juiciness) so an increasing R value is indicative of water loss and a decreasing R value is indicative of water accumulation as well as measured Xc is proportional to cell mass so a decreased Xc is indicative of cell membrane loss through such processes (naturally occurring or artificially induced) as fragmentation or proteolysis; a diminution of the Xc value and/or a change in the rate of said diminution from a zenith towards a nadir is indicative of optimal palatability (tenderness) which may progress beyond that nadir of palatability and become non-palatable. Comparison of the Xc value of one sample of the same genus and species, section and cut of a biological entity to another sample of the same genus and species, section and cut of a biological entity illustrates a comparative scale of palatability. A consumer may have a subjective selection of a particular palatability scale value which translates to his/her individual desire and preference.

The present invention also provides a method of palatability assessment of a foodstuff biological entity being assessed, comprising the steps of: placing signal introduction and detection electrodes on/in or/around the foodstuff subject studied such as, on or within the opposite lateral peripheral borders of said organ upon selecting or harvesting of said biological entity; placing signal introduction and detection electrodes on/in or/around or within the superior and inferior borders of said biological entity for a first part of an initial measurement upon said selection and harvesting of said biological entity; measuring and recording the first values of impedance, resistance and reactance and calculating capacitance and phase angle of said biological entity in said initial measurement; then placing said signal introduction and detection electrodes on/in or/around or within said superior and said inferior borders of said biological entity; placing said signal introduction and detection electrodes on/in or/around or within said opposite lateral borders of said biological entity; measuring and recording second values of said impedance, resistance and said reactance and calculating capacitance and phase angle of said of said biological entity; and comparing said first and/or second values to normal, average, optimal and individual values to determine the scale of palatability of said biological entity and by serial measurements if said palatability has changed in response to time (aging), external intervention (chemical, electrical or mechanical) or not for and then serially additional series of said measurements and calculations are repeated at predetermined intervals based upon the individual characteristics of the biological entity, the time it was harvested and the manner it is stored and transported.

Alternative electrode scheme arrays include alternative external placements to include: circumferential wrapping, multiple placement locations and placement of the study subject on any such array.

Yet another alternative is the internal placement of an electrode array in which the electrodes are introduced into the study subject at various locations, depths and configurations.

Yet another variation in measurement is the entry or placement of the study subject within an electrical field (such as generated within a solenoid) and through a fixed or scanning process measures the electrical properties as related to the water and cell content as they relate to palatability.

One embodiment of the invention is the assessment and illustration of the aging process to provide objective and subjective scaling to price, sell and market based on results.

Another embodiment of the present invention is to grade and report such palatability values for the purpose of pricing and salability in a grocery.

Another embodiment of the invention is a sales and marketing tool by presenting palatability as a menu/product variable available from a merchant, such as a meat producer, grocer or restaurateur.

Yet another embodiment of the invention would be utilization by the consumer at home, point of purchase or point in time of preparation or consumption in the assessment of palatability of foodstuffs.

Yet another embodiment of the invention would be as part of an external appliance such as a scale, refrigerator, display or packaging system or portable temperature-controlled appliance.

Yet another embodiment of the invention is the determination when the foodstuff is not palatable, safe or unsafe.

A specific purpose of the invention is in its application to the following example; a sub-primal loin cut section is removed two days after harvest (post-mortem) from a USDA Premium Choice beef carcass during in-plant fabrication.

The tenderloin sub-primal while hanging has four stainless steel electrode quality skewer probes placed through it, the first and outer pair at the beginning (top) and end (bottom) of the loin, becoming the BIA signal introduction electrodes and within that first pair a second pair is placed to the approximated beginning and end of the ‘strip loin’ longissimus dorsi becoming the BIA signal detection electrodes, the impedance foodstuff plethysmograph is connected to the electrodes, energized and the readings of resistance and reactance are taken, automatically entered identified, date and time-stamped into the instrument the impedance foodstuff plethysmograph is disengaged and the electrodes probes removed and calculations of impedance, capacitance and phase angle are made and converted into a corresponding value of a palatability index for that specific cut of beef (in this instance a four point five on an acceptable range of from three to six) and reported.

Throughout the twenty-eight day aging process selected for this cut the measurement procedure is repeated every four days for sixteen days (four measurements that can coincide with the transit of the meat from processor, to purveyor to merchant provider; retail grocer or restaurateur) and the newly determined values are compared to the initial values to establish the rate of change and the rate of continued testing, every other day or every day based on progression towards the optimal value range for this cut at which time the meat is available for final sale, disposition, processing and preparation and consumption as a end-user consumer may select their individual subjective preference value from the determined palatability index (in this instance a final index value of nine, with a premium tenderness range of from seven to ten).

Although the invention has been described in detail in the foregoing only for the purpose of illustration, it is to be understood that such detail is solely for that purpose and that variations can be made therein by those of ordinary sill in the art without departing from the spirit and scope of the invention as defined by the following claims, including all equivalents thereof.

1. A method of determining and monitoring palatability of a foodstuff biological entity including at least a portion a a live or previously-live organism, comprising the steps of:
subjecting said foodstuff biological entity to bioelectrical impedance analysis for measurement and composition analysis; and
utilizing results of said subjecting step to illustrate an objective scale of palatability of said foodstuff biological entity.

2. A method according to claim 1, wherein:
said bioelectrical impedance analysis includes measurement and/or calculation of resistance, reactance, impedance, capacitance, and/or phase angle of said foodstuff biological entity.

3. A method according to claim 1, wherein:
said utilizing step also determines a value of a “Palatability Index” for said foodstuff biological entity.

4. A method according to claim 2, including:
placing said foodstuff biological entity or a portion thereof in an electrical field; and
taking said measurements through a fixed or scanning process.

5. A method according to claim 1, wherein:
said bioelectrical impedance analysis includes measurement and/or calculation of resistance, reactance, impedance, capacitance, and/or phase angle of said foods biological entity as determined through measurement by mono or multiple frequencies or spectroscopic analysis and by series and/or parallel circuit models; and
using voltage and current sufficient to accommodate the geometry of said foodstuff biological entity.

6. A method according to claim 1, wherein:
said foodstuff biological entity includes meat;
said method is utilized to determine optimal aging, curing, and/or processing of said meat; and
said subjecting step includes subjecting said meat to bioelectrical impedance analysis for measurement, composition analysis, and serial tracking and grading of said aging of said meat and the determination of aging (intentional or incidental) beyond palatability.

7. A method according to claim 3, wherein:
said foodstuff biological entity, includes meat;
said method is utilized to determine optimal aging, curing, and/or processing of said meat; and
said subjecting step includes subjecting said meat to bioelectrical impedance analysis for measurement, composition analysis, and serial tracking and grading of said aging of said meat and the determination of aging (intentional or incidental) beyond palatability.

8. A method according to claim 6, including:
placing said foodstuff biological entity or a portion thereof in an electrical field; and
taking said measurements through a fixed or scanning process.

9. A method according to claim 6, wherein:
said bioelectrical impedance analysis includes measurement and/or calculation of resistance, reactance, impedance, capacitance, and/or phase angle of said meat and foodstuff, and
including the step of comparing said measurements and calculations to normal values, average values, optimal values, and/or individual values, and in response to time and/or external influences purposeful or incidental.

10. A method according to claim 7, wherein:
said bioelectrical impedance analysis includes measurement and/or calculation of resistance, reactance, impedance, capacitance, and/or phase angle of said meat and foodstuff; and
including the step of comparing said measurements and calculations to normal values, average values, optimal values, and/or individual values, and in response to time and/or external influences purposeful or incidental.

11. A method according to claim 7, including:
placing said foodstuff biological entity or a portion thereof in an electrical field; and
taking said measurements through a fixed or scanning process.

12. A method according to claim 6, wherein:
said bioelectrical impedance analysis includes measurement and/or calculation of resistance, reactance, impedance, capacitance, and/or phase angle of said meat as determined through measurement by mono or multiple frequencies or spectroscopic analysis, and
including the step of comparing said measurements and calculations to normal values, average values, optimal values, and/or individual values, and in response to time and/or external influences purposeful or incidental.

13. A method for determining palatability of a foodstuff biological entity, changes of palatability of said biological entity, and/or timing of optimal palatability, loss of the palatability of said biological entity and/or illustrating an objective scale of palatability from which a producer, purveyor, merchant, preparer or consumer may objectively and subjectively apply individual tastes and select from said scale their preference, comprising the steps of:
providing normal, average, optimal and individual measured values of resistance, reactance, capacitance and phase angle, of a sample subject studied of said foodstuff biological entity;
measuring initial values of impedance, resistance, reactance, capacitance and phase angle of said sample subject foodstuff biological entity;
taking measurements of impedance, resistance, reactance, capacitance and phase angle, at predetermined intervals of time based upon said sample subject foodstuff biological entity;
recording said measurements;
comparing initial values of said measurements to normal values of said measurements and to serially measured values of said measurements;
determining, from said comparison steps, hallmarks of palatability of said foodstuff biological entity, said progression of changes in palatability of said biological entity, to a specific individual Palatability Index value which may be reported and found as the inherent average, normal, optimal and/or safe individual characteristics of said foodstuff biological entity or portion thereof.

14. A method according to claim 13, including:
measuring initial values of impedance, resistance, reactance, capacitance and phase angle of said sample subject biological entity as determined by mono or multiple frequencies or spectroscopic analysis and/or at various current, voltage and power, and by series and/or parallel circuit models requirements as to accommodate the inherent characteristics of the foodstuff biological entity.

15. A method according to claim 13, including:
placing said foodstuff biological entity or a portion thereof in an electrical field; and
taking said measurements through a fixed or scanning process.

16. A method according to claim 13, including the steps of
determining a first value of a “Palatability Index” from said measured initial values of impedance, resistance, reactance, capacitance and phase angle of said sample subject foodstuff biological entity;
determining a second value of said “Palatability Index” from said measurements at said predetermined intervals of time; and
determining third values of said “Palatability Index” based upon said comparison steps.

17. A method of palatability assessment for a foodstuff biological entity being assessed, comprising the steps of:
measuring and recording first values of impedance, resistance and reactance and calculating capacitance and phase angle of said foodstuff biological entity in an initial measurement and;
placing said signal introduction and detection electrodes on/in or/around or within the superior and said inferior borders of said foodstuff biological entity;
placing said signal introduction and detection electrodes on/in or/around or within the opposite lateral borders of said foodstuff biological entity;
measuring and recording second values of said impedance, resistance and said reactance and calculating capacitance and phase angle of said of said biological entity;
comparing said first and/or second values to normal, average, optimal and individual values to determine if said foodstuff biological entity is palatable or not; and
performing serially additional series of said measurements and calculations repeated at predetermined intervals based upon individual characteristics of said foodstuff biological entity, the time it was harvested, and the manner it is stored and transported.

18. A method according to claim 17, including:
placing signal introduction and detection electrodes on/in or/around or within said foodstuff biological entity or a portion thereof, such as, on or within said opposite lateral peripheral borders of said foodstuff biological entity upon selecting or harvesting of said biological entity; and
placing signal introduction and detection electrodes on/in or/around or within said foodstuff biological entity at superior and inferior borders of said foodstuff biological entity or said portion thereof for a first part of said initial measurement upon said selection and harvesting of said foodstuff biological entity.

19. A method according to claim 17, including:
placing signal introduction and detection electrodes on, in or around or within said foodstuff biological entity or a portion thereof by placing said foodstuff biological entity or a portion thereof onto an electrode configuration comprised singularly or as part of an external appliance, such as, on or within the opposite lateral peripheral borders of said foodstuff biological entity or a portion thereof upon selecting or harvesting of said foodstuff biological entity; and
placing signal introduction and detection electrodes on, in or around or within said foodstuff biological entity or a portion thereof by placing said foodstuff biological entity or a portion thereof onto an electrode configuration singularly or as part of an external appliance at on, around or within the superior and inferior borders of said foodstuff biological entity or said portion thereof for a first part of said Initial measurement upon said selection and harvesting of said foodstuff biological entity.

20. A method according to claim 17, including:
placing said foodstuff biological entity or a portion thereof in an electrical field; and
taking said measurements through a fixed or scanning process.
